(19) United States
(12) Patent Application Publication
(10) Pub. No.: US 2025/0002677 A1
(43) Pub. Date: Jan. 2, 2025

(54) PROCESSES FOR RECOVERING DIALKYL TEREPHTHALATES FROM FEEDSTOCKS

(71) Applicant: Eastman Chemical Company, Kingsport, TN (US)

(72) Inventors: Pinguan Zheng, Johnson City, TN (US); Perry Michael Murdaugh, Johnson City, TN (US); Hannah Grace Harris, Kingsport, TN (US)

(73) Assignee: Eastman Chemical Company, Kingsport, TN (US)

(21) Appl. No.: 18/703,842

(22) PCT Filed: Oct. 25, 2022
(86) PCT No.: PCT/US2022/047651

§ 371 Date: Apr. 23, 2024

Related U.S. Application Data
(60) Provisional application No. 63/262,977, filed on Oct. 25, 2021.

(51) Int. Cl. C08J 11/24; C07C 31/20; C07C 43/13; C07C 69/82; C08J 11/08; C08J 11/16

(52) U.S. Cl. CPC C08J 11/24; C07C 31/202; C07C 43/132; C07C 69/82; C08J 11/08; C08J 11/16; C08J 2367/02

(57) ABSTRACT

Processes for recovering dialkyl terephthalates. The processes can include exposing a polyester composition to one or more glycols to depolymerization conditions thereby providing one or more depolymerization products. The one or more depolymerization products can be exposed to an alcoholysis process to recover dialkyl terephthalate.

### FIELD OF THE INVENTION

The present disclosure relates to processes for recycling one or more polyesters in a feedstock composition. More particularly, the present disclosure relates to recovering dialkyl terephthalates from feedstock compositions.

### BACKGROUND OF THE INVENTION

Certain conventional systems may utilize glycolysis and/or methanolysis processes in an attempt to recycle polyesters. However, certain conventional glycolysis and/or methanolysis processes may require a substantial amount of resources and energy in order to arrive at suitable products for use in subsequent production processes, e.g., production processes to generate recycled polyesters or other compositions.

### BRIEF SUMMARY OF THE INVENTION

In one aspect, a process for recovering one or more dialkyl terephthalates from a feedstock composition is provided. The process can include exposing a feedstock composition comprising one or more polyesters and one or more foreign materials to one or more glycols and a depolymerization catalyst in a first reaction vessel under depolymerization conditions to provide a first mixture. The first mixture can include one or more depolymerization products. The process can also include exposing at least a portion of the first mixture to an alcohol composition and an alcoholysis catalyst under alcoholysis conditions to provide a second mixture. The second mixture can include one or more dialkyl terephthalates. The alcoholysis conditions can include a temperature of from 23° C. to 70° C. for 0.5 hours to 10 hours. The process can also include isolating at least a portion of the one or more dialkyl terephthalates from the second mixture.

### BRIEF DESCRIPTION OF THE DRAWING

FIG. 1 is an example system for recovering one or more dialkyl terephthalates from a feedstock composition, in accordance with aspects of the present disclosure.

### DETAILED DESCRIPTION OF THE INVENTION

### Overview

The present disclosure may be understood more readily by reference to the following detailed description of certain aspects of the disclosure and working examples. In according with the purpose(s) of this disclosure, certain aspects of the disclosure are described in the Brief Summary of the Invention and are further described herein below. Also, other aspects of the disclosure are described herein.

Aspects herein are directed to processes for recovering one or more dialkyl terephthalates from feedstock compositions. As described herein, an example process can include exposing a feedstock composition to one or more glycols under depolymerization conditions to generate one or more depolymerization products, which are then exposed to an alcoholysis process, followed by an isolation of the dialkyl terephthalate.

As discussed above, certain conventional glycolysis and/or methanolysis processes may require a substantial amount of resources and energy in order to arrive at suitable products for use in subsequent production processes, e.g., production processes to generate recycled polyesters or other compositions.

The processes and systems disclosed herein can alleviate one or more of the above problems. For instance, in certain aspects, the processes disclosed herein can include exposing a polyester composition to depolymerization conditions with one or more glycols to provide one or more depolymerization products. In various aspects, the one or more depolymerization products can include monomers, oligomers, or a combination thereof. In aspects, the one or more depolymerization products can be exposed to alcoholysis conditions resulting in a dialkyl terephthalate product of high yield and purity. As discussed herein, the alcoholysis conditions include a temperature that is reduced compared to certain conventional systems, which reduces the overall energy and resources required. In aspects, as discussed herein, the depolymerization and alcoholysis conditions described herein are substantially milder than certain conventional processes, which results in less ethylene glycol yield loss, e.g., due to fewer side reactions or degradation reactions converting ethylene glycol into various impurities. Further, in certain aspects as discussed further below, glycols present in the resulting alcoholysis liquid component can be separated from at least a portion of the alcohol composition utilized in the alcoholysis, and these recycle glycols can be re-used in subsequent rounds of dialkyl terephthalate recovery, which also reduces resource consumption.

### Feedstock Compositions

In various aspects, the feedstock compositions for the processes disclosed herein comprise one or more polyesters and one or more foreign materials.

The term “polyester” can refer to a synthetic polymer prepared by the reaction of one or more difunctional carboxylic acids and/or multifunctional carboxylic acids with one or more difunctional hydroxyl compounds and/or multifunctional hydroxyl compounds. The difunctional carboxylic acid can be a dicarboxylic acid and the difunctional hydroxyl compound can be a dihydric alcohol such as, for example, glycols. Furthermore, as used herein, the term “diacid” or “dicarboxylic acid” includes multifunctional acids, such as branching agents. The term “glycol” or “diol” as used herein, includes, but is not limited to, diols, glycols, and/or multifunctional hydroxyl compounds. The dicarboxylic acid residues may be derived from a dicarboxylic acid monomer or its associated acid halides, esters, salts, anhydrides, or mixtures thereof. As used herein, therefore, the term dicarboxylic acid is intended to include dicarboxylic acids and any derivative of a dicarboxylic acid, including its associated acid halides, esters, halfesters, salts, half-salts, anhydrides, mixed anhydrides, or mixtures thereof, useful in a reaction process with a diol to make polyester. It should be understood, that the term “polyester” as used herein also refers to copolyesters.

As used herein, the term “residue(s)” refers to the monomer unit or repeating unit in a polymer, oligomer, or dimer. For example, a polymer can be made from the condensation of the following monomers: terephthalic acid (“TPA”) and cyclohexyl-1,4-dimethanol (“CHDM”). The condensation reaction results in the loss of water molecules. The residues in the resulting polymer are derived from either terephthalic acid or cyclohexyl-1,4-dimethanol. Below in Formula (I), a non-limiting example of a polyester is provided.

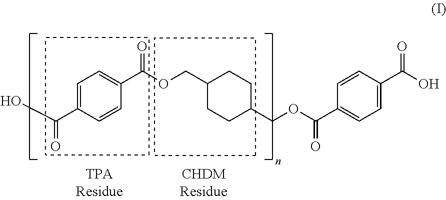

In aspects, the polyesters exhibit an inherent viscosity of from about 0.1 dL/g to about 1.2 dL/g as determined according to ASTM D2857-70, about 0.2 dL/g to about 1.2 dL/g as determined according to ASTM D2857-70, about 0.3 dL/g to about 1.2 dL/g as determined according to ASTM D2857-70, or about 0.4 dL/g to about 1.2 dL/g as determined according to ASTM D2857-70.

In various aspects, the one or more polyesters can include terephthalate polyesters. Terephthalate polyesters are polyesters that comprise residues of terephthalic acid or residues of any derivative of terephthalic acid, including its associated acid halides, esters, half-esters, salts, half-salts, anhydrides, mixed anhydrides, and/or mixtures thereof or residues thereof useful in a reaction process with a diol to make a copolyester In various aspects, the polyesters can include polyethylene terephthalate (PET). In one or more aspects, the polyester can include glycol-modified PET. In certain aspects, the polyester can include polyethylene terephthalate (PET), 1,4-cyclohexanedimethanol (CHDM)-modified PET, isophthalic acid (IPA)-modified PET, diethylene glycol (DEG)-modified PET, glycol-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene) glycol (PEG)-modified PET, polycyclohexylenedimethylene terephthalate (PCT), cyclohexanedimethanol (CHDM)-containing copolyester, isosorbide-containing copolyester, or a combination thereof. In the same or alternative aspect, the polyester composition can include polyethylene terephthalate (PET) that comprises CHDM, IPA, DEG, NPG, PDO, BDO, HDO, MP diol, isosorbide, PTMG, PEG, or a combination thereof.

In various aspects, the polyesters can include CHDM. In one aspect, the polyesters can include about 0 mole % to about 100 mole % CHDM, about 1 mole % to about 100 mole % CHDM, about 1 mole % to about 90 mole % CHDM, about 1 mole % to about 80 mole % CHDM, about 1 mole % to about 70% CHDM, about 1 mole % to about 60 mole % CHDM, about 1 mole % to about 50 mole % CHDM, about 1 mole % to about 40 mole % CHDM, about 1 mole % to about 35 mole % CHDM, about 1 mole % to about 30 mole % CHDM, about 1 mole % to about 25 mole % CHDM, about 1 mole % to about 20 mole % CHDM, about 1 mole % to about 10 mole % CHDM, or about 1 mole % to about 5 mole % CHDM. In aspects, the mole % of CHDM refers to the mole % of CHDM relative to all diol equivalents in the polyesters. In various aspects, the polyester can include DEG. In aspects, the polyesters can include about 0 mole % to about 100 mole % DEG, about 1 mole % to about 100 mole % DEG, about 1 mole % to about 90 mole % DEG, about 1 mole % to about 80 mole % DEG, about 1 mole % to about 70 mole % DEG, about 1 mole % to about 60 mole % DEG, about 1 mole % to about 50 mole % DEG, about 1 mole % to about 40 mole % DEG, about 1 mole % to about 35 mole % DEG, about 1 mole % to about 30 mole % DEG, about 1 mole % to about 20 mole % DEG, about 1 mole % to about 10 mole % DEG, about 1 mole % to about 5 mole % DEG, or about 1 mole % to about 3 mole % DEG. In aspects, the mole % of DEG refers to the mole % of DEG relative to all diol equivalents in the polyesters. In aspects, the polyesters can include isophthalic acid. In aspects, the polyesters can include about 0 mole % to about 30 mole % isophthalic acid, about 1 mole % to about 30 mole % isophthalic acid, about 1 mole % to about 25 mole % isophthalic acid, about 1 mole % to about 20 mole % isophthalic acid, about 1 mole % to about 15 mole % isophthalic acid, about 1 mole % to about 10 mole % isophthalic acid, about 1 mole % to about 7.5 mole % isophthalic acid, about 1 mole % to about 5 mole % isophthalic acid, about 1 mole % to about 3 mole % isophthalic acid, about 10 mole % or less of isophthalic acid, about 7.5 mole % or less of isophthalic acid, about 5 mole % or less of isophthalic acid, or about 3 mole % or less of isophthalic acid. In aspects, the mole % of isophthalic acid refers to the mole % of isophthalic acid relative to all diacid equivalents in the polyesters. In certain aspects, the polyesters can include about 0 mole % to about 100 mole % CHDM, about 0 mole % to about 100 mole % DEG, about 0 mole % to about 30 mole % isophthalic acid, or a combination thereof. In certain aspects, the polyesters can include about 1 mole % to about 100 mole % CHDM, about 1 mole % to about 100 mole % DEG, about 1 mole % to about 30 mole % isophthalic acid, or a combination thereof. In various aspects, the polyesters can include other glycols, e.g., other than those mentioned above. For instance, in aspects, the polyesters can include, but is not limited to, neopentyl glycol (NPG), 2-methyl-2,4-pentanediol (MP diol), butanediol (BDO), propanediol (PDO), hexanediol (HDO), isosorbide, poly(tetramethylene ether) glycol (PTMG), poly(ethylene) glycol (PEG), or a combination thereof. In certain aspects, each of the NPG, MP diol, BDO, PDO, HDO, isosorbide, PTMG, and PEG can be present in the polyesters in an amount of 0 mole % to about 100 mole %, about 1 mole % to about 100 mole %, about 1 mole % to about 90 mole %, about 1 mole % to about 80 mole %, about 1 mole % to about 70%, about 1 mole % to about 60 mole %, about 1 mole % to about 50 mole %, about 1 mole % to about 40 mole %, about 1 mole % to about 35 mole %, about 1 mole % to about 30 mole %, about 1 mole % to about 25 mole %, about 1 mole % to about 20 mole %, about 1 mole % to about 10 mole %, or about 1 mole % to about 5 mole %. In aspects, the mole % of each of NPG, MP diol, BDO, PDO, HDO, isosorbide, PTMG, and PEG refers to the mole % of each of NPG, MP diol, BDO, PDO, HDO, isosorbide, PTMG, and PEG, respectively, relative to all diol equivalents in the polyesters. In various aspects, the polyesters can include CHDM, DEG, NPG, MP diol, BDO, PDO, HDO, isosorbide, PTMG, PEG, isophthalic acid, or a combination thereof, where each component is present in any of the amounts for such components described in this paragraph.

In aspects, the polyester composition or the one or more polyesters present in the polyester composition can be recycled polyesters. In various aspects, the recylcled polyester(s) can include material that was recovered as manufacturing scrap, industrial waste, post-consumer waste, or a combination thereof. In aspects, the recylced polyester(s) can be prior-used products that have been used and/or discarded. In aspects, the polyester composition and/or recycled polyester(s) can come from various sources and/or in various forms, including but not limited to textiles, carpet, thermoformed materials, bottles, pellets, and film.

In various aspects, as discussed above, the feedstock composition can include one or more foreign materials. In certain aspects, the foreign materials can be any non-polyester material. In one or more aspects, the one or more foreign materials may include, but are not limited to, polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron, or a combination thereof. In various aspects, the one or more foreign materials can be present in the feedstock composition in an amount of from about 0.01 wt. % to about 50 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 7.5 wt. %, about 0.01 wt. % to about 5 wt. %, about 0.01 wt. % to about 2.5 wt. %, about 0.01 wt. % to about 1.0 wt. %, relative to the weight of the one or more polyesters in the polyester composition.

In aspects, the feedstock composition can be in solid form, liquid form, molten form, or in a solution. In certain aspects, the solution can include one or more polyesters pre-dissolved in a solvent, e.g., DMT, EG, DEG, TEG, or a combination thereof.

### Optional Pre-Treatment of the Polyester Composition

In certain aspects, an optional treatment of the feedstock composition, prior to glycolysis and/or methanolysis, can be performed. In various aspects, the optional pretreatment can include any type of treatment that aids in removing a portion of any foreign materials from the feedstock composition and/or that aids in recovering one or more polyesters from a mixed feedstock, e.g., a feedstock comprising the foreign materials discussed above. For instance, in one aspect, the optional pretreatment can include exposing to the feedstock composition to one or more solvents, in an effort to selectively dissolve the polyester in the feedstock composition (or at least a portion of the foreign materials in the feedstock composition) to allow for separation between at least a portion of the foreign materials and the one or more polyesters in the feedstock composition. As one example aspect, the optional pretreatment can include exposing the feedstock composition to one or more solvents, e.g., one or more solvents that can cause dissolution of the polyester in the feedstock composition. For instance, the one or more solvents can include but are not limited to 4-methylcyclohexanemethanol (MCHM), ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether) glycol (PTMG), dibutyl terephthalate (DBT), dioctyl terephthalate (DOTP), ethylene carbonate (EC), dimethyl carbonate (DMC), dimethyl sulfoxide (DMSO), dimethylformamide (DMF), or combinations thereof. In the same or alternative aspects, the feedstock composition can be exposed to the one or more solvents at specific temperatures to effectuate dissolution of one or more components. In various aspects, a pretreatment process can include one or more dissolution and separation steps using various solvents and/or temperatures to achieve a desired level of foreign materials removal and/or purity level of PET. For instance, in one aspect, a dissolution and separation can be utilized using one solvent at a specific temperature, e.g., to remove one or more foreign materials, followed by a subsequent dissolution and separation of the polyester fraction using another solvent at a specific temperature, e.g., to remove one or more other foreign materials. The dissolution and/or separation(s) in this optional pretreatment step can utilize any suitable systems, reactors, vessels, and/or separation techniques to achieve a desired pretreated feedstock composition.

### Glycolysis of the Feedstock Composition

As discussed above, in various aspects, the processes disclosed herein can include exposing a feedstock composition to depolymerization conditions to depolymerize at least a portion of the one or more polyesters into one or more depolymerization products. In various aspects, the one or more depolymerization products can include monomers, oligomers, or a combination thereof. In certain aspects, the oligomers can exhibit a degree of polymerization from 2 to 10, 2 to 8, 2 to 6, or 2 to 4. In aspects, the one or more polyesters may be depolymerized into one or more depolymerization products that can include monomers and terephthalate oligomers having a degree of polymerization from 2 to 10, 2 to 8, 2 to 6, or 2 to 4. In aspects, liquid chromatography can be utilized to discern the degree of polymerization of an oligomer, and/or gel permeation chromatography can be utilized to discern the molecular weight of the oligomers.

In aspects, the term degree of polymerization (DP) can refer to the number of residues in the oligomer. As used herein, the degree of polymerization (DP) refers to the number of difunctional carboxylic acid residues and/or multifunctional carboxylic acid residues in the oligomer. For instance, in one example aspect, a DP of one, would refer to a residue that includes one terephthalic acid residue or one isophthalic acid residue. In such an example aspect, a DP of one can also be referred to as a monomer. A non-limiting example of a DP of one is provided below in formula (II).

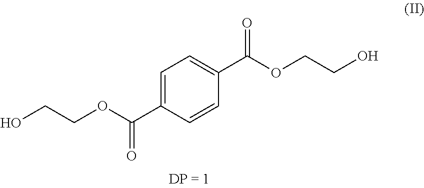 [SMILES: O=C(OCCO)c1ccc(C(=O)OCCO)cc1]

Formulas (III)-(V) below show non-limiting examples of oligomers having a DP of two, three, and n, respectively, in aspects.

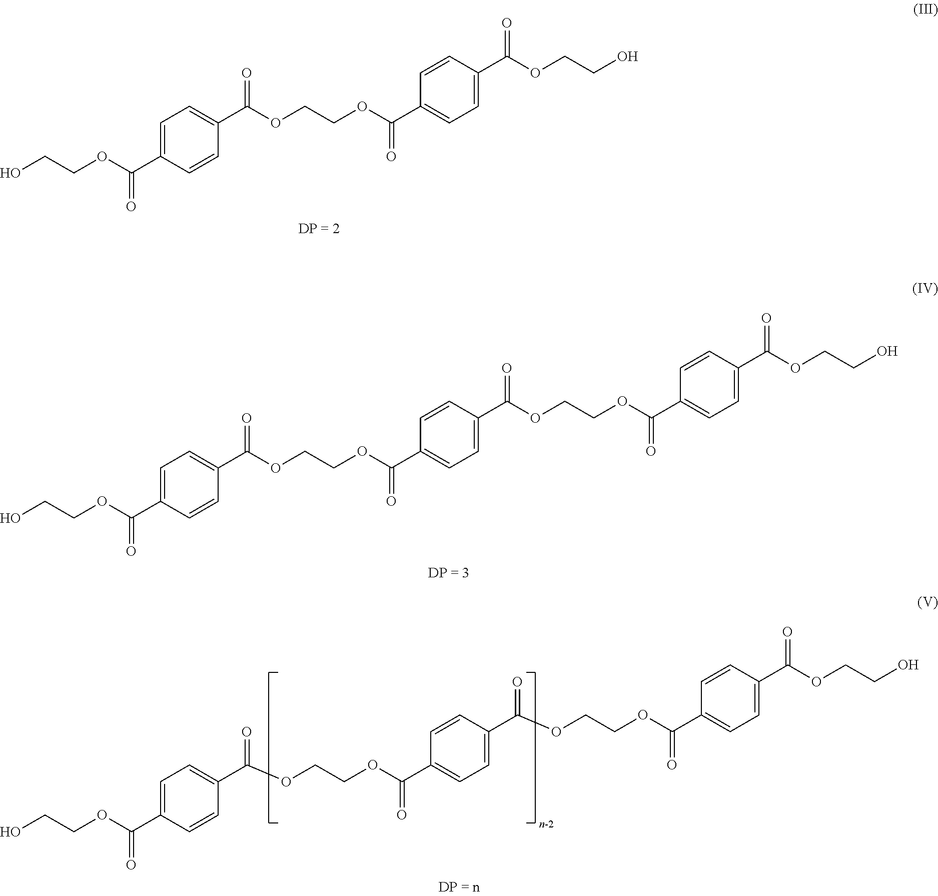

In aspects, this depolymerization can occur via a glycolysis process. Generally, in aspects, the glycolysis process can include exposing a feedstock composition to one or more glycols, where the glycols react with the polyester, optionally in the presence of a trans-esterification catalyst, forming a mixture of bis(hydroxyethyl) terephthalate (BHET) and low molecular weight terephthalate oligomers. Some representative examples of glycolysis methods are disclosed in U.S. Pat. Nos. 3,257,335; 3,907,868; 6,706,843; and 7,462,649, and are incorporated by reference herein.

In one aspect of a glycolysis process, one or more polyesters, e.g., one or more recycled polyesters, and one or more glycols can be fed into a glycolysis reactor where the one or more recylced polyesters are dissolved and depolymerized under depolymerization conditions.

In aspects, any amount of the one or more glycols suitable for use in a glycolysis process can be utilized. In various aspects, the weight ratio of the one or more glycols relative to the amount of the polyester composition can be of from 12:1 to 1:12, 8:1 to 1:9, 6:1 to 1:9, 4:1 to 1:9, 4:1 to 1:7, 4:1 to 1:4, 4:1 to 1:2, 3:1 to 1:9, 3:1 to 1:7, 3:1 to 1:4, 3:1 to 1:2, 2:1 to 1:9, 2:1 to 1:7, 2:1 to 1:4, 2:1 to 1:2, 4:1 to 2:7, 3:1 to 1:4, 3:1 to 1:3, 2:1 to 1:2, 2:1 to 3:7, 1:1 to 3:7, 4:1 to 3:7, 4:1 to 4:7, 4:1 to 5:7, or 3:1 to 3:7.

In certain aspects, the one or more glycols can include any glycol suitable for use in a glycolysis process. As used herein, the term “glycol” refers to aliphatic, alicyclic, and aralkyl glycols. Exemplary glycols include ethylene glycol, 1,2-propandiol (also known propylene glycol), 1,3-propanediol, 1,4-butanediol, 1,5-pentanediol, 1,6-hexanediol, 2,2-dimethyl-1,3-propanediol, 1,2-cyclohexane dimethanol, 1,3-cyclohexane dimethanol, 1,4-cyclohexane dimethanol, 2,2,4,4-tetramethyl-1,3-cyclobutanediol, isosorbide, p-xylylenediol, and the like. These glycols may also contain ether linkages, such as is the case in, for example, diethylene glycol, triethylene glycol, and tetraethylene glycol. Additional embodiments of glycols include higher molecular weight homologs, known as polyethylene glycols, such as those produced by Dow Chemical Company under the Carbowax™ tradename. In one embodiment, the polyethylene glycol has a molecular weight of from greater than 200 to about 10,000 Daltons (Mn). These glycols also include higher order alkyl analogs, such as dipropylene glycol, dibutylene glycol, and the like. Similarly, further glycols include higher order polyalkylene ether diols, such as polypropylene glycol and polytetramethylene glycol with molecular weights ranging from about 200 to about 10,000 Daltons (Mn) (also referred to as g/mole). In one aspect, the glycol can be chosen from aliphatic, alicyclic, and aralkyl glycols. In the same or alternative aspects, the glycol can be chosen from ethylene glycol; 1,2-propandiol; 1,3-propanediol; 1,4-butanediol; 1,5-pentanediol; 1,6-hexanediol; 2,2-dimethyl-1,3-propanediol; 1,2-cyclohexane dimethanol; 1,3-cyclohexane dimethanol; 1,4-cyclohexane dimethanol; 2,2,4,4-tetramethyl-1,3-cyclobutanediol; isosorbide; p-xylylenediol; diethylene glycol; triethylene glycol; tetraethylene glycol; polyethylene glycols; dipropylene glycol; dibutylene glycol; polyalkylene ether diols chosen from polypropylene glycol and polytetramethylene glycol.

In various aspects, the one or more glycols can include ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether) glycol (PTMG), or a combination thereof. In one aspect, the one or more glycols can include about 0 wt. % to about 100 wt. % EG, or about 1 wt. % to about 100 wt. % EG, relative to the total weight of the one or more glycols. In certain aspects, the one or more glycols can include about 0 wt. % to about 100 wt. % DEG, or about 1 wt. % to about 100 wt. % DEG, relative to the total weight of the one or more glycols. In certain aspects, the one or more glycols can include about 0 wt. % to about 100 wt. % TEG, or about 1 wt. % to about 100 wt. % TEG, relative to the total weight of the one or more glycols. In certain aspects, the one or more glycols can include about 0 wt. % to about 100 wt. % PEG, or about 1 wt. % to about 100 wt. % PEG, relative to the total weight of the one or more glycols. In certain aspects, the one or more glycols can include about 0 wt. % to about 100 wt. % NPG, or about 1 wt. % to about 100 wt. % NPG, relative to the total weight of the one or more glycols. In certain aspects, the one or more glycols can include about 0 wt. % to about 100 wt. % PDO, or about 1 wt. % to about 100 wt. % PDO, relative to the total weight of the one or more glycols. In certain aspects, the one or more glycols can include about 0 wt. % to about 100 wt. % BDO, or about 1 wt. % to about 100 wt. % BDO, relative to the total weight of the one or more glycols. In certain aspects, the one or more glycols can include about 0 wt. % to about 100 wt. % MP diol, or about 1 wt. % to about 100 wt. % MP diol, relative to the total weight of the one or more glycols. In certain aspects, the one or more glycols can include about 0 wt. % to about 100 wt. % PTMG, or about 1 wt. % to about 100 wt. % PTMG, relative to the total weight of the one or more glycols. In aspects, the one or more glycols can include about 0 wt. % to about 50 wt. % CHDM, or about 1 wt. % to about 50 wt. % CHDM, relative to the total weight of the one or more glycols. In one aspect, the one or more glycols can include 0 wt. % to about 100 wt. % EG, 0 wt. % to about 100 wt. % DEG, 0 wt. % to about 100 wt. % TEG, 0 wt. % to about 100 wt. % PEG, 0 wt. % to about 100 wt. % NPG, 0 wt. % to about 100 wt. % PDO, 0 wt. % to about 100 wt. % BDO, 0 wt. % to about 100 wt. % MP diol, 0 wt. % to about 100 wt. % PTMG, and 0 wt. % to about 50 wt. % CHDM, relative to the total weight of the one or more glycols. In one aspect, the one or more glycols can include 1 wt. % to about 100 wt. % EG, 1 wt. % to about 100 wt. % DEG, 1 wt. % to about 100 wt. % TEG, 1 wt. % to about 100 wt. % PEG, 1 wt. % to about 100 wt. % NPG, 1 wt. % to about 100 wt. % PDO, 1 wt. % to about 100 wt. % BDO, 1 wt. % to about 100 wt. % MP diol, 1 wt. % to about 100 wt. % PTMG, and 1 wt. % to about 50 wt. % CHDM, relative to the total weight of the one or more glycols. In certain aspects, as discussed in detail below, the one or more glycols can be recycle glycols that were recovered from a prior glycolysis and methanolysis process for recovery of one or more dialkyl terephthalates, as disclosed herein.

In various aspects, as discussed above, the glycolysis process can include one or more catalysts, e.g., trans-esterification catalysts. In certain aspects, the catalyst can be present in an amount of from 0.1 wt. % to 10 wt. %, relative to the weight of the polyester composition. In aspects, any suitable catalyst can be utilized. In one aspect, the catalyst can include a carbonate catalyst, for example, but not limited to: Li₂CO₃, K₂CO₃, Na₂CO₃, Cs₂CO₃, ZrCO₃, or a combination thereof. In one aspect, the catalyst can include a hydroxide catalyst, for example, but not limited to: LiOH, NaOH, KOH, tetrabutylammonium hydroxide (TBAH), or a combination thereof. In one aspect, the catalyst can include an alkoxide catalyst, for example, but not limited to: sodium methoxide (NaOMe), lithium methoxide (LiOMe), magnesium methoxide, potassium t-butoxide, ethylene glycol monosodium salt, ethylene glycol disodium salt, or a combination thereof. In one aspect, the catalyst can include tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), and manganese (II) acetate (Mn(OAc)₂)), or a combination thereof. In certain aspects, the catalyst can include LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), and zinc acetylacetonate hydrate (Zn(acac)₂), or a combination thereof. In one aspect, the catalyst can include LiOH, NaOH, KOH, sodium methoxide (NaOMe), and lithium methoxide (LiOMe). In certain aspects, the catalyst can include Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, lithium chloride, or a combination thereof.

The depolymerization conditions can include a temperature of from 150° C. to 260° C. and an absolute pressure of from 1 atmosphere (atm) to 15 atm, or 1 atm to 2 atm, in an agitated reactor for 0.5 h to 10 h. Higher temperatures may be used to increase the rate of depolymerization; however, reactor systems that can withstand elevated pressures may be required. One or a plurality of reactors may be used for the reaction of the polyester with the one or more glycols. For example, the reaction mixture can be continuously withdrawn from the first stage and introduced to a second stage maintained under pressure, along with additional glycol, wherein depolymerization continues to the desired degree of completion. In various aspects, any type of vessel, reactor, and/or reactor system can be utilized for the depolymerization or glycolysis of the polyester composition. In one aspect, a continuous stirred-tank reactor or vessel, a fixed bed reactor, or a melt extruder. In the same or alternative aspects, the depolymerization or glycolysis of the polyester composition can be a batch or continuous process.

In various aspects, exposing the feedstock composition to the depolymerization conditions described herein may result in one or more components in the feedstock composition, e.g., one or more foreign materials, melting and/or forming a gel-like layer within the reaction vessel and may float on the top or near the top of the solvent or glycols in the reaction vessel. In one example aspect, foreign materials including polyolefins, polyethylene, polypropylene, polystyrene, or a combination thereof, may float on the top or near the top of the solvent or glycols in the reaction vessel, under the depolymerization conditions described herein. In such aspects, these foreign materials may be removed from the glycolysis reaction vessel using any suitable removal process, e.g., via a pump or other process.

As discussed further below, the liquid component is further subjected to at least a methanolysis process for the recovery of one or more dialkyl terephthalates. In various aspects, prior to exposing the resulting mixture from the depolymerization or glycolysis process to the alcoholysis process described below, the resulting mixture can optionally be allowed to cool to a temperature of about 150° C. or less, or of from about 50° C. to about 150° C. In aspects, the resulting mixture can be allowed to cool to the desired temperature in the glycolysis reaction vessel(s) or can be transferred to a different vessel for temperature reduction.

### Alcoholysis of the One or More Depolymerization Products

As discussed above, in aspects, the one or more depolymerization products produced in the glycolysis process described above can be subjected to an alcoholysis process. In certain aspects the glycolysis process can provide a first mixture that can include the one or more depolymerization products, e.g., monomers and/or oligomers having a degree of polymerization of from 2 to 10, along with the one or more glycols, catalysts, at least a portion of foreign materials, or a combination thereof. In one aspect, the first mixture from the glycolysis process may be subjected to the alcoholysis process without intervening separation processes.

Generally, in a typical alcoholysis process, a polyester is reacted with an alcohol, e.g., methanol, to produce a depolymerized mixture comprising oligomers, terephthalate monomers, e.g., dimethyl terephthalate (DMT), and one or more glycols. In other embodiments, other monomers such as, for example, CHDM, DEG, and dimethyl isophthalate (DMI), also may be produced, depending on the composition of the polyester. In one embodiment, during the alcoholysis process the terephthalate oligomers are reacted with methanol to produce a depolymerized polyester mixture comprising polyester oligomers, DMT, CHDM, and/or EG.

Some representative examples of the methanolysis of PET are described in U.S. Pat. Nos. 3,321,510; 3,776,945; 5,051,528; 5,298,530; 5,414,022; 5,432,203; 5,576,456; 6,262,294; which are incorporated herein by reference.

In aspects, the alcoholysis process can include exposing the first mixture including the one or more depolymerization products resulting from the glycolysis process to an alcohol composition under conditions resulting in one or more dialkyl terephthalates. As discussed above, in aspects, the one or more depolymerization products can be present in the liquid component resulting from the glycolysis process. Without being bound by any particular theory, it is believed that since the glycolysis process is performed using a lower amount of glycols compared to certain conventional processes (or a weight ratio of glycols relative to the amount of the polyester composition of from 3:1 to 1:9) this allows for the resulting liquid component from the glycolysis process to be directly utilized in the alcoholysis process without requiring further processing, e.g., to concentrate the resulting one or more depolymerization products and/or remove a portion of the glycols.

The alcohol composition can include any suitable alcohol known in the art for use in an alcoholysis process to obtain a specific dialkyl terephthalate. In one aspect, the alcohol composition can be methanol. In aspects, when methanol is utilized as the alcohol composition, DMT can be the resulting methanolysis product.

In certain aspects, the amount of the alcohol composition can be any amount that is in excess on a weight basis relative to the amount or weight of the polyester composition. In certain aspects, a weight ratio of the amount of the alcohol composition relative to the amount of the polyester composition can be from about 2:1 to about 10:1. In such aspects, the amount of the polyester composition refers to the amount or weight of the polyester composition that is utilized in the above glycolysis process.

In aspects, the alcoholysis reaction can occur at a temperature of about 90° C. or less, about 80° C. or less, about 70° C. or less, about 60° C. or less, about 50° C. or less, about 40° C. or less, or about 30° C. or less. In the same or alternative aspects, the alcoholysis reaction can occur at a temperature of from about 20° C. to about 90° C., about 20° C. to about 80° C., about 20° C. to about 70° C., about 20° C. to about 60° C., about 20° C. to about 50° C., about 20° C. to about 40° C., or about 20° C. to about 30° C. In various aspects, without being bound by any particular theory, it is believed that, since in the processes disclosed herein, the polyester in the polyester composition has already undergone at least a partial depolymerization process, e.g., in the glycolysis step discussed above, that the methanolysis process can be performed at the temperatures described above, which are comparably reduced compared to certain other conventional processes. Additionally or alternatively, without being bound by any particular theory, it is believed that, since the one or more depolymerization products produced in the glycolysis process are separated from the waste or insoluble material prior to this alcoholysis process, the alcoholysis process can be conducted at the reduced temperatures described above.

In aspects, the alcoholysis process can be conducted in any suitable reactor and/or vessel. In aspects, the alcoholysis reactor can be in fluid communication with the reactor utilized in the glycolysis process described above. In certain aspects, the alcoholysis reactor is a different reactor than the vessel used for glycolysis. Alternatively, in various aspects, the alcoholysis process can be conducted in the same vessel as the glycolysis process and/or the filtration process discussed above. In certain aspects, the alcoholysis process can be conducted at ambient pressure, e.g., about 1 atm, or at a pressure of from about 1 atm to about 5 atm, or of from about 1 atm to about 3 atm. In various aspects, the alcoholysis reaction can be conducted at a pressure above ambient pressure, e.g., more than 1 atm, or about 5 atm or less, about 3 atm or less, when the alcoholysis reaction temperature is high for the process conditions disclosed herein, e.g., about 50° C. or more, about 60° C. or more, about 70° C. or more, about 80° C. or more, or about 90° C. or more.

In various aspects, an alcoholysis catalyst can be utilized in the alcoholysis process. In aspects, the alcoholysis catalyst can be present in an amount of from about 0.1 wt. % to about 20 wt. % relative to the weight of the polyester composition, or of from about 0.1 wt. % to about 10 wt. % relative to the weight of the polyester composition, or of from about 0.1 wt. % to about 5 wt. % relative to the weight of the polyester composition, or of from about 0.1 wt. % to about 2 wt. % relative to the weight of the polyester composition, or of from about 0.1 wt. % to about 1 wt. % relative to the weight of the polyester composition, or of from about 0.1 wt. % to about 0.5 wt. % relative to the weight of the polyester composition. In such aspects, the amount of the polyester composition refers to the amount or weight of the polyester composition that is utilized in the above glycolysis process. In various aspects, the alcoholysis catalyst amounts disclosed in this paragraph refer to the amount of alcoholysis catalyst present during the alcoholysis reaction. In various aspects, the alcoholysis catalyst amounts disclosed in this paragraph refer to the amount of alcoholysis catalysts that is added to one or more depolymerization products and the one or more alcohols to facilitate the alcoholysis reaction. In certain aspects, reduced or lower amounts of alcoholysis catalyst may be added to the one or more depolymerization products and the one or more alcohols to facilitate the alcoholysis reaction, such as an amount of from about 0.1 wt. % to about 10 wt. % relative to the weight of the polyester composition, or of from about 0.1 wt. % to about 5 wt. % relative to the weight of the polyester composition, or of from about 0.1 wt. % to about 2 wt. % relative to the weight of the polyester composition, or of from about 0.1 wt. % to about 1 wt. % relative to the weight of the polyester composition, or of from about 0.1 wt. % to about 0.5 wt. % relative to the weight of the polyester composition. In aspects, such a lower amount of alcoholysis catalysts may be added at least partly because alcoholysis catalyst is already present in the one or more depolymerization products and/or one or more alcohols. In such aspects, as discussed below, the alcohol and/or glycol may be recycled and re-used in subsequent glycolysis and alcoholysis process as disclosed herein, which may include at least a portion of alcoholysis catalyst from a prior alcoholysis and/or glycolysis process.

In various aspects, the alcoholysis catalyst can include a carbonate catalyst, for example, but not limited to: K₂CO₃, Na₂CO₃, Li₂CO₃, Cs₂CO₃; a hydroxide catalyst, for example, but not limited to: KOH, LiOH, NaOH; an alkoxide catalyst, for example, but not limited to NaOMe, Mg(OMe)₂, KOMe, KOt-Bu, ethylene glycol monosodium salt, ethylene glycol disodium salt, or a combination thereof. In certain aspects, the alcoholysis catalyst can include KOH, NaOH, LiOH, or a combination thereof. In certain aspects, the alcoholysis catalyst can include NaOMe, KOMe, Mg(OMe)₂, KOt-Bu, ethylene glycol monosodium salt, ethylene glycol disodium salt, or a combination thereof. In various aspects, the alcoholysis catalyst can be in solid form, a solution form in water, methanol, or ethylene glycol, or a combination of thereof. In certain aspects, the alcoholysis catalyst can be added to the one or more depolymerization products and the alcohol composition once the alcohol composition and the one or more depolymerization products reach the desired reaction temperature or temperature range disclosed above.

The one or more depolymerization products can be exposed to the alcohol composition and optionally the alcoholysis catalyst under the temperature and pressure conditions described above for a period of time to achieve the desired yield of the resulting dialkyl terephthalate. In certain aspects, the one or more depolymerization products can be exposed to the alcohol composition and optionally the alcoholysis catalyst under the temperature and pressure conditions described above for a period of time of from about 5 minutes to about 5 hours, or of from about 5 minutes to about 2 hours, or about 5 minutes to about 1 hour, or about 5 minutes to about 30 minutes, or about 5 minutes to about 15 minutes, or about 5 minutes to about 10 minutes.

In aspects, the alcoholysis process results in a mixture that includes one or more dialkyl terephthalates. In various aspects, the alcoholysis process results in a mixture wherein the dialkyl terephthalate is an insoluble and/or solid component, which may also include one or more foreign materials. Isolation of the DMT is discussed further below. In aspects, the liquid component of this resulting mixture from the glycolysis process can include glycols, the alcohol composition, DEG, CHDM, or a combination thereof. In one aspect, the glycols can be the glycols that were utilized in the glycolysis process and present with the one or more depolymerization products at the initiation of the alcoholysis process. In various aspects, the crude dialkyl terephthalate product can be isolated from the mixture using any known separation technique, e.g., filtering, centrifugation, sedimentation, settling, or a combination of one or more separation techniques. In aspects, the filtering may include washing the solid component with additional alcohol composition or other solvent. The resulting liquid component can include the filtrate and wash.

As discussed above, the resulting crude dialkyl terephthalate product can include the dialkyl terephthalate as well as one or more foreign materials or other insoluble materials generated in the depolymerization and alcoholysis process described herein. In aspects, the dialkyl terephthalate can be isolated using any suitable isolation process. In one aspect, the dialkyl terephthalate can be isolated using distillation processes, dissolution processes, or both. In certain aspects, optionally, prior to the distillation processes and/or dissolution processes, the crude product may be subjected to melting, filtering, washing, or a combination thereof. In certain aspects, the crude product can be exposed to any suitable distillation conditions for isolation of the dialkyl terephthalate. The distillation can occur in any vessel or distillation system that is suitable for use in the processes and systems described herein. In various aspects, the distillation process of the crude product can result in a pot residue that may comprise one or more foreign materials from the feedstock. In the same or alternative aspects, the crude product may be subjected to one or more dissolution processes using a solvent, wherein the solvent has Hansen Solubility Parameters (HSPs) of a total solubility parameter of δ_{T}=16±3. In such aspects, solvent may be, but is not limited to, ethyl acetate, isopropyl acetate, isobutyl acetate, n-butyl acetate, n-propyl acetate, butyl propionate, Eastman PM acetate, isobutyl isobutyrate, n-pentyl propionate, mixed hexyl acetate esters, 2-ethylhexyle acetate, Eastman EB acetate, ethylene glycol acetate, and Eastman Texanol™. In one aspect, the solvent may be a linear ether and/or a cyclic ether, including but not limited to 1,4-dioxane, Eastman EB solvent, Eastman EP solvent. In one aspect, solvent may be a ketone, and/or hydrocarbon, including, but not limited to toluene, methyl ethyl ketone. In one aspect, more than one dissolution and solid-liquid separation may be utilized to remove one or more of the foreign materials to result in a more pure product. In certain aspects, dissolution may occur at 150° C. or less, 120° C. or less, 100° C. or less, 80° C. or less, 60° C. or less. In certain aspects, the weight ratio of solvent to crude DMT may be 50% or less, 40% or less, 30% or less, 20% or less, 10% or less. In some aspects, solvent may be recycled and re-used by distillation. In various aspects, the DMT product may be isolated as solid.

In various aspects, the isolated dialkyl terephthalate can include about 90 wt. % or more dialkyl terephthalate, e.g., DMT, about 93 wt. % or more dialkyl terephthalate, e.g., DMT, or about 95 wt. % or more dialkyl terephthalate, e.g., DMT, relative to the weight of the isolated component. In the same or alternative aspects, the dialkyl terephthalate, e.g., DMT, in the resulting isolated dialkyl terephthalate component can be about 90% or more pure, about 93% or more pure, or about 95% or more pure. In various aspects, the resulting isolated dialkyl terephthalate component can also include dimethyl isophthalate (DMI). In such aspects, the DMI can be present in an amount of about 1000 ppm or less, or about 500 ppm or less, or of from about 1 ppm to about 1000 ppm, or of from about 1 ppm to about 500 ppm. In one or more aspects, the resulting isolated dialkyl terephthalate component can also include bisphenol A (BPA). In such aspects, the BPA can be present in an amount of about 1000 ppm or less, or about 500 ppm or less, or of from about 1 ppm to about 1000 ppm, or of from about 1 ppm to about 500 ppm.

The processes described herein, e.g., the glycolysis and/or alcoholysis processes are substantially mild compared to certain conventional processes, e.g., high temperature one-step glycolysis or methanolysis processes. For instance, certain conventional one-step processes may utilize a glycolysis process at temperatures of 240° C. or above in the presence of a Lewis acid catalyst, for instance, Zn(OAc)₂ or KOAc. Such harsh conditions can result in reduced EG yield from the depolymerization, as the EG is converted in various side reactions to various impurity compounds, including but not limited to: diethylene glycol (DEG), triethylene glycol (TEG), acetaldehyde, 1,1-dimethoxyethane, 1,2-dimethoxyethane, dioxane, 2-methoxyethanol, 1-methoxyethanol, and dimethyl ether. In aspects, the processes described herein are substantially milder than such conventional processes, and also result in less EG yield loss, e.g., from less side reactions converting EG into various impurities. In one aspect, the processes described herein result in about 5% or less yield loss of EG, about 2% or less yield loss of EG, or about 1% or less yield loss of EG, or about 0.5% or less yield loss of EG. In such aspects, the yield loss of EG is the percent of EG that is formed into impurities, e.g., DEG, relative to the combined amount of EG from the polyester composition feed and of the EG added in the glycolysis process. In the same or alternative aspects, the processes described herein result in minimal glycol impurities being produced. For instance, in one aspect, the processes described herein can result in the net generation of about 5 wt. % or less DEG, about 2 wt. % or less DEG, or about 1 wt. % or less DEG, or about 0.5 wt. % or less DEG, or of from about 0.01 wt. % to about 5 wt. % DEG, about 0.01 wt. % to about 2 wt. % DEG, or about 0.01 wt. % to about 1 wt. % DEG, or about 0.01 wt. % to about 0.5 wt. % DEG, or about 0.01 wt. % to about 0.2 wt. % DEG, when EG is used as the one or more glycols in the glycolysis process. In aspects, the processes described herein can result in the net generation of about 5 wt. % or less DEG and/or other impurity, about 2 wt. % or less DEG and/or other impurity, or about 1 wt. % or less DEG and/or other impurity, or about 0.5 wt. % or less DEG and/or other impurity, or of from about 0.01 wt. % to about 5 wt. % DEG and/or other I impurity, about 0.01 wt. % to about 2 wt. % DEG and/or other impurity, or about 0.01 wt. % to about 1 wt. % DEG and/or other impurity, or about 0.01 wt. % to about 0.5 wt. % DEG and/or other impurity, or about 0.01 wt. % to about 0.2 wt. % DEG and/or other impurity, when EG is used as the one or more glycols in the glycolysis process. In aspects, the net generation of DEG (or other impurity) is the weight percent of the amount of DEG or other impurity that is present over the amount of DEG or other impurity present in the polyester composition feed. In one aspect, the DEG being produced can be produced in the glycolysis process described herein and/or the alcoholysis process described herein. In certain aspects, the EG and/or any glycol impurities, such as DEG when using EG as the one or more glycols in the glycolysis process, can be present in the resulting liquid component from this alcoholysis step. In certain aspects, utilization of a Lewis base catalyst, e.g., a hydroxide-based or carbonate-based catalyst, in the glycolysis process may also facilitate or contribute to reduced EG degradation and/or a reduction of glycol impurities.

### Recycling Glycols

As discussed above, in various aspects, the glycols utilized in the glycolysis process can be re-used in subsequent rounds of processes for recovery of one or more dialkyl terephthalates disclosed herein. At a high level, in aspects, the liquid component resulting from the alcoholysis process can be processed for re-use, e.g., for re-use in subsequent rounds of glycolysis of a subsequent polyester composition to recover one or more dialkyl terephthalates.

In aspects, as discussed above, the liquid component resulting from the alcoholysis process can include glycols, the alcohol composition, DEG, CHDM, or a combination thereof. In aspects, the glycols in this liquid component can be the glycols that were utilized in the glycolysis process and present with the one or more depolymerization products at the initiation of the alcoholysis process. In various aspects, this liquid component can be subjected to a separation process, e.g., to remove or separate at least a portion of the alcohol composition, for instance, methanol, or a mixture of methanol and ethylene glycol. In certain aspects, for removal of at least a portion of the alcohol composition, the liquid component can be exposed to distillation or short path distillation. In such aspects, the distillation conditions can include exposing the liquid component to a temperature of about 220° C. or less, about 200° C. or less, about 180° C. or less, about 160° C. or less, about 150° C. or less, about 130° C. or less, about 60° C. or more, about 70° C. or more, of from about 60° C. to about 220° C., of from about 70° C. to about 220° C., of from about 60° C. to about 180° C., or of from about 60° C. to about 160° C. In the same or alternative aspects, the distillation conditions can include a pressure of from about 1 Torr (133.3 Pa) to about 800 Torr (106,657 Pa), or about 30 Torr (3999 Pa) to about 500 Torr (66,661 Pa). In aspects, the liquid component can be exposed to the distillation conditions until all or a substantial portion of the alcohol composition has been removed, e.g., vaporized, from the liquid component. In certain aspects, at least a portion of the alcohol composition, if present with the recycle glycols, may be removed during a subsequent glycolysis process, e.g., may be removed or vaporized due to the glycolysis conditions.

In aspects, the distillation of the liquid component can occur in any vessel or distillation system that is suitable for use in the processes and systems described herein. In one aspect, the distillation vessel can be in fluid communication with the alcoholysis reaction vessel and/or any component of the filtering process utilized subsequent to the alcoholysis, e.g., to isolate the dialkyl terephthalate solid or insoluble component. In the same or alternative aspects, the distillation vessel can be in fluid communication with the glycolysis vessel.

In various aspects, the distillation of the liquid component can cause the alcohol composition to vaporize leaving a pot residue. In aspects, the pot residue includes the glycols and any other heavies, e.g., non-vaporizable compounds present in the liquid component. In aspects, the glycols in the pot residue can be referred to as recycle glycols and/or the glycols from a non-vaporizable portion of a continuous distillation process using the distillation conditions described herein can be referred to as recycle glycols.

In aspects, as discussed above, the recycle glycols can be utilized in a subsequent round of the process described herein to recover one or more dialkyl terephthalates from a polyester composition. Further, in aspects, the recycle glycols can be recycled again using the process described herein, after going through this subsequent round of dialkyl terephthalate recovery. In aspects, the recycle glycols can be recovered and re-used at least two, at least three, at least four, or at least five times. In certain aspects, when the recycle glycols are used in subsequent round(s) of dialkyl terephthalate recovery, addition of a catalyst in the subsequent glycolysis step(s) may be omitted, as the recycle glycols may include prior-used catalyst.

In aspects, when the recycles glycols are recovered and re-used, it has been unexpectedly found that the resulting dialkyl terephthalates recovered exhibit comparable purity to that of dialkyl terephthalates recovered using glycols that have not been recovered and re-used. This comparable purity of the dialkyl terephthalate is present, in aspects, after re-using recycle glycols at least two times, at least three times, at least four times, or at least five times resulting in a dialkyl terephthalate recovery having a purity of at least about 90%, at least about 93%, or at least about 95%.

As discussed above, in aspects, the processes described herein are substantially milder than certain conventional processes, and also result in less EG yield loss, e.g., due to fewer side reactions converting EG into various impurities. In one example with three EG recycle experiments, the yield loss of EG to DEG is about 5% or less, about 2% or less, about 1% or less, or about 0.5% or less. In one example with four EG recycle experiments, the yield loss of EG to DEG is about 5% or less, about 2% or less, about 1% or less, or about 0.5% or less. In the same or alternative aspects, the processes described herein result in minimal glycol impurities being produced. In certain aspects, the EG and/or any glycol impurities, such as DEG when using EG as the one or more glycols in the glycolysis process, can be present in the resulting liquid component from the alcoholysis process discussed above. In such aspects, the DEG or any glycol impurities can be recovered and/or present in the recycle glycols described herein. In such aspects, the recycle glycols can include about 5 wt. % or less DEG and/or other impurity, about 2 wt. % or less DEG and/or other impurity, or about 1 wt. % or less DEG and/or other impurity, or about 0.5 wt. % or less DEG and/or other impurity, or of from about 0.01 wt. % to about 5 wt. % DEG and/or other impurity, about 0.01 wt. % to about 2 wt. % DEG and/or other impurity, or about 0.01 wt. % to about 1 wt. % DEG and/or other impurity, or about 0.01 wt. % to about 0.5 wt. % DEG and/or other impurity, or about 0.01 wt. % to about 0.2 wt. % DEG and/or other impurity, when EG is used as the one or more glycols in the glycolysis process.

### Use of Recovered Dialkyl Terephthalates to Form Polyesters or Other Products

As discussed above, the processes disclosed herein can result in high purity dialkyl terephthalates, such as DMT. For instance, in certain aspects, the recovered DMT can be utilized to form one or more polyesters, including but not limited to PET and TMCD-containing polyesters. In various aspects, the products formed using the recovered DMT may be indistinguishable from similar products formed from virgin DMT. In such aspects, any suitable process for forming the PET and TMCD-containing polyesters can be utilized, since the DMT is of sufficient purity.

In the same or alternative aspects, the recovered DMT can be utilized to form CHDM. In various aspects, the CHDM formed using recovered DMT may be indistinguishable from CHDM formed from virgin DMT, due to the high purity of the recovered DMT. In such aspects, the CHDM can be formed from the recovered DMT using any suitable process.

### Example Systems

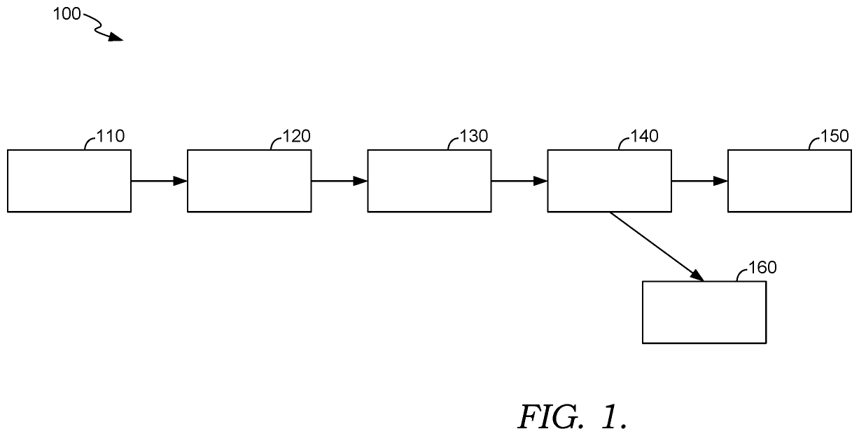

FIG. 1 schematically depicts one example system and/or process for recovering one or more dialkyl terephthalates from a feedstock composition. The system 100 includes a source 110 of feedstock composition, e.g., the feedstock composition described above. The vessel 120 represents the glycolysis vessel, where the feedstock composition is received and exposed to one or more glycols under depolymerization conditions, as discussed in detail above. In aspects, the vessel 120 can be in fluid communication with the source 110. In various aspects, as discussed above, the feedstock composition, after exposure to the depolymerization conditions in the vessel 120, is converted into one or more depolymerization products. In various aspects, as discussed above, the one or more depolymerization products can include monomers and/or oligomers having a degree of polymerization of from 2 to 10, 2 to 8, 2 to 6, or 2 to 4. As discussed, in certain aspects, one or more of the foreign materials may be removed from the glycolysis vessel, e.g., foreign materials that may melt and/or float on the top of the glycols or solvent in the glycolysis vessel. In the aspect depicted in FIG. 1, the one or more depolymerization products and/or the resulting mixture from the glycolysis process can be exposed to alcoholysis conditions in a vessel 130. In aspects, the one or more depolymerization products and/or the liquid component can be directly utilized in this alcoholysis process. Alcoholysis conditions are discussed in detail above. In aspects, as discussed above, the alcoholysis of the one or more depolymerization products and/or the liquid component can result in a mixture that includes an insoluble or solid component that comprises the crude dialkyl terephthalate and a liquid component that comprises the alcohol composition, glycols, and potentially other soluble components as described herein. As discussed above, the resulting alcoholysis reaction mixture can be exposed to a solid-liquid separation device 140, e.g., a filtering system, to separate the solid component containing the crude dialkyl terephthalate product. In aspects, as discussed above, the filtrate or liquid component can be subjected to one or more distillation or other processes at the system 150 to recover glycols and/or methanol, which may optionally be returned to the glycolysis reaction or alcoholysis reactor, respectively. As described above, the crude dialkyl terephthalate product may undergo more or more isolation processes at the system 160, e.g., distillation and/or dissolution, to provide dialkyl terephthalate product of high purity. In certain aspects, the process described herein associated with the system 100 can be performed as a continuous process, a batch process, or a semi-continuous process. It is understood that the system 100 is just one example system and other configurations of system components are contemplated by the disclosure herein. For instance, one or more of the components of the system 100 may not be physically separated, or distinct, from one or more other components of the system 100. It is further understood that the system 100 is only schematically depicted in order to highlight aspects of the processes disclosed herein.

The present disclosure can be further illustrated by the following examples of aspects thereof, although it will be understood that these examples are included merely for purposes of illustration and are not intended to limit the scope of the disclosure unless otherwise specifically indicated.

### EXAMPLES

### Materials

Solid-state blend (SSB) contains 84.265% TPA and 1.599% IPA by LC analysis. Theoretical 86.4% TPA for PET sample.
25% PCR contains 82.103% TPA and 1.563% IPA by LC analysis.
FDST-251 contains 77.157% TPA and 1.511% IPA by LC analysis.
Ethylene glycol, methanol, potassium carbonate and 50% sodium hydroxide aqueous solution were obtained from Sigma Aldrich. All chemicals and reagents were used as received, unless otherwise mentioned.

### Analytical Procedures

Gas Chromatography (GC) Analysis. GC analysis was performed on an Agilent model 7890B gas chromatograph equipped with a 7693A autosampler and two G4513A towers. The gas chromatograph (GC) was outfitted with two columns—a 60 m×0.32 mm×1.0 micron DB-1701™ (J&W 123-0763) and a 60 m×0.32×1 micron DB-1™ (J&W 123-1063)—and samples were injected simultaneously onto both columns. A shared oven temperature program was used, and sample components were detected by flame ionization detection (FID). Five-point calibrations were performed for components of interest. The gas chromatograph was interfaced to an EZChrom Elite Chromatography Data System. Methanolysis product samples were prepared by adding a known volume of pyridine-based internal standard solution to a known mass of sample and then derivatizing with N,O-Bis(trimethylsilyl) trifluoroacetamide (BSTFA).

Gel Permeation Chromatography (GPC). Size exclusion chromatography GPC analysis was performed on an Agilent series 1100 GPC/SEC analysis system with a UV-Vis detector. The column set used was Polymer Laboratories 5 μm Plgel, with guard, mixed C and oligopore. The eluent consists of 95% methylene chloride and 5% hexafluoroisopropanol with tetraethylammonium nitrate (1 gram/2 liter solvent). The testing was performed at ambient temperature with a flow rate of 1.0 mL/min. Instrument was calibrated with linear PET oligomer standard. Sample was prepared by dissolving 10 mg sample in 10 mL methylene chloride/hexafluoroisopropanol (70/30). 10 μL Toluene was added as flow rate marker. The injection volume was 10 μL. BHET (bis(hydroxyethyl) terephthalate) GC wt %, MHT (4-(methoxycarbonyl) benzoic acid) GC wt. %, MHET (Methyl-2-hydroxyethyl terephthalate) GC wt. %, and dimethyl isophthalate (DMI) GC wt. % are provided as a read-out from the GC process software.

Liquid Chromatography (LC). LC analysis for oligomers was performed on an HP 1100 series liquid chromatograph equipped with diode array detector (DAD) with a range of 190-900 nm. The system was fitted with a Zorbax Poroshell 120 EC-C18 (4.6×50 mm, 2.7 μm) column at 40° C. The flow rate was 1.0 mL/min. Mobile phases were water (25 nM ammonium acetate) (A) and acetonitrile (B). The elution gradient was as follows: 0 min, 95% A/5% B; 2 min, 95% A/5% B; 18 min, 0% A/100% B; 28 min, 0% A/100% B; 28.1 min, 95% A/5% B; 33 min, 95% A/5% B. Sample solution was prepared by dissolving ˜4 mg sample in 1 mL DMF/DMSO (50/50). The injection volume was 2 μL. Oligomer distribution was reported as area %.

LC analysis for TPA and IPA was performed on an HP 1100 series liquid chromatograph equipped with a fluorescence detector using excitation wavelength of 225 nm, emission wavelength of 310 nm, a FLD PMT gain of 10 and data frequency of 2.31 Hz. The system was fitted with an Agilent Poroshell EC-C18 (4.6×150 mm, 2.7 μm) column at 30° C. Mobile phases were 0.14% phosphoric acid in water (A), acetonitrile (B) and THF (C). The elution gradient was as follows: 0 min, 79% A/0% B/21% C; 10 min, 79% A/0% B/21% C; 18 min, 34% A/45% B/21% C; 18.1 min, 14% A/65% B/21% C; 19 min, 14% A/65% B/21% C; 19.1 min, 79% A/0% B/21% C; 25 min, 79% A/0% B/21% C. The flow rate was 0.9 mL/min. TPA and IPA content was reported wt %.

XRF Quantitative Metal Analysis. Quantitative XRF testing was carried out using Malvern Panalytical Zetium WDXRF. DMT and Feedstock samples were prepared by compressing them into chips. All samples were analyzed in a helium mode with 4 μm Mylar film attached. This method can detect 14 key elements with quantification ranging from several to hundreds of ppm w/w.

DMT yield % was calculated as: (weight of final DMT)/(theoretical DMT weight)*100%.

DMT GC purity % was calculated as: (weight % of DMT in final product by GC)/(total wt. % by GC)*100%.

Inherent Viscosity Measurement. The Inherent viscosities (IV) of the particular polymer materials useful herein are determined according to ASTM D2857-70 procedure, in a Wagner Viscometer of Lab Glass, Inc., having a ½ mL capillary bulb, using a polymer concentration about 0.5% by weight in 60/40 by weight of phenol/tetrachloroethane. The procedure is carried out by heating the polymer/solvent system at 120° C. for 15 minutes, cooling the solution to 25° C. and measuring the time of flow at 25° C. The IV is calculated from the equation:

$${η}_{inh} = \frac{ln \frac{{t}_{S}}{{t}_{0}}}{C}$$

where: η: inherent viscosity at 25° C. at a polymer concentration of 0.5 g/100 ml of solvent; t_{S}: sample flow time; t₀: solvent-blank flow time; C: concentration of polymer in grams per 100 mL of solvent. The units of the inherent viscosity throughout this application are in the deciliters/gram.

In the following examples, a viscosity was measured in tetrachloroethane/phenol (50/50, weight ratio) at 30° C. and calculated in accordance with the following equation:

$${η}_{inh} = \frac{ln ( {η}_{sp} )}{C}$$

wherein ηₛₚ is a specific viscosity and C is a concentration.

### Example 1: Direct Low Temperature Methanolysis of High Molecular Weight Feedstock SSB to DMT

In this Example 1, a 3-necked 1-liter round-bottom flask was equipped with a mechanical stirrer, a reflux condenser, and a thermocouple. Charge SSB dissolver sample (100.65 g) and methanol (401.37 g). The resulting mixture was heated to 64° C. at 500 rpm agitation. Once the reaction reached the set temperature, 50% aqueous NaOH solution (468 mg) was added. 20 minutes later, the mixture was cooled to 50° C. and 2^{nd} dose NaOH solution (468 mg) was added. 1 hour later, a 3^{rd} dose NaOH solution was added. The resulting mixture was stirred for another hour. The flask was allowed to cool to room temperature and product was recovered by filtration and wash. Insoluble solid was also isolated as solid chunk by carefully decanting the slurry. After drying in the air for overnight, product was obtained 49.57 g as grey powder, which contained 49.9% DMT by GC analysis. Insoluble was isolated (40.5 g). LC analysis showed that insoluble solid contained 76.08% TPA, 1.387% IPA. GPC analysis gave a Mn of 1051, Mw of 1525 and Mz of 2002.

### Example 2: Glycloysis and Low Temperature Methanolysis of High Molecular Weight Feedstock SSB to DMT

In this Example, a 3-necked 1-liter round-bottom flask was equipped with a mechanical stirrer, a reflux condenser, and a thermocouple. Charge SSB dissolver sample (97.94 g, from pilot plant dissolver), EG (42.53 g) and K₂CO₃ (0.97 g). The resulting mixture was refluxed for 2 hours. After the reaction mixture was cooled to 50° C., 50% aqueous NaOH solution (468 mg) and MeOH (400.29 g) were added. Then the mixture was cooled to 50° C. and 2^{nd} dose NaOH solution (612 mg) was added. The resulting mixture was kept at 50° C. and stirred for 30 minutes. The flask was allowed to cool to room temperature and product was recovered by filtration and wash. After drying in the air for overnight, DMT product was obtained 70.19 g as grey powder (92.45% GC purity).

### Example 3: Converting PCR Feedstock to DMT with Methanolysis at Reflux

A 3-necked 1-liter round-bottom flask was equipped with a mechanical stirrer, a reflux condenser, and a thermocouple. Charge 25% PCR carpet dissolver sample (100.11 g) and methanol (408.95 g). The resulting mixture was heated to reflux. Once the reaction reached the set temperature, 50% aqueous NaOH solution (468 mg) was added and hold for 1 hour. Then the mixture was cooled to 50° C. and 2^{nd} dose NaOH solution (468 mg) was added. 30 minutes later, the flask was allowed to cool to room temperature and product was recovered by filtration and wash. Insoluble solid was also isolated by carefully decanting the slurry as solid chunk (65.94 g). DMT product was obtained as grey solid (24.83 g, 59.5% GC purity).

TABLE 1
DMT Analysis from Examples 1-3

| | Dissolver | | mass balance % | | |
|---|---|---|---|---|---|
| | sample | Process | Insoluble | DMT | GC purity % |
| Example 1 | SSB | methanolysis | 40.2% | 49.2% | 49.9% |
| Example 3 | 25% PCR | methanolysis | 65.8% | 24.8% | 59.5% |
| Example 2 | SSB | Glycolysis; then methanolysis | n/d | 71.6% | 92.5% |

As can be seen in Table 1, direct methanolysis of the feedstocks from Examples 1 and 3 at low temperature delivered DMT product with low mass balance and low GC purity. In addition, a significant amount of insoluble solid was isolated by careful decanting and analyzed to be high MW PET oligomer for Examples 1 and 3. The presence of high MW PET oligomer and broad MW distribution may contribute to low yield and low purity of isolated DMT product in these Examples. A glycolysis step prior to methanolysis, as in Example 2, was demonstrated to improve the yield and GC purity. The result confirmed the presence of high MW PET oligomer and that the conversion into lower MW oligomer may facilitate an efficient methanolysis.

### Example 4: Convert Feedstock to DMT Through Glycolysis Using a PET/EG Ratio of 7/3

A 3-necked 1-liter round-bottom flask was equipped with a mechanical stirrer, a reflux condenser, and a thermocouple. Charge SSB sample (87.51 g), EG (37.69 g) and K₂CO₃ (0.90 g). The resulting mixture was refluxed for 2 hours. After the reaction mixture was cooled to 50° C., 50% aqueous NaOH solution (546 mg) and MeOH (350.8 g) were added. The resulting mixture was kept at 50° C. and stirred for 30 minutes. The flask was allowed to cool to room temperature and product was recovered by filtration and wash. After drying in the air for overnight, DMT product was obtained 32.27 g as grey powder (85.81% GC purity). Solid chunk (51.01 g) was also isolated and mainly consisted of PET oligomer.

### Example 5: Convert Feedstock to DMT Through Glycolysis Using a PET/EG Ratio of 1/1 Followed by Short Path Distillation

A 3-necked 1-liter round-bottom flask was equipped with a mechanical stirrer, a reflux condenser, and a thermocouple. Charge SSB sample (100.18 g), EG (103.38 g) and K₂CO₃ (1.01 g). The resulting mixture was refluxed for 3 hours. After the reaction mixture was cooled to 50° C., 50% aqueous NaOH solution (546 mg) and MeOH (403.7 g) were added. The resulting mixture was kept at 50° C. and stirred for 30 minutes. The flask was allowed to cool to room temperature and product was recovered by filtration and wash. After drying in the air for overnight, DMT product was obtained 90.07 g as grey powder (95.73% GC purity).

A 3-necked 1-liter round bottom flask was equipped with a magnetic stir bar, a heating mantle, a distillation head and an air condenser, and a thermocouple. Both distillation head and air condenser were insulated with a heat tape and a thermocouple. Charge 121.89 g crude DMT. DMT distillation was carried out at 44.4 to 46.3 torr vacuum and 186.3° C. take-off temperature. DMT product was collected as white needle (112.15 g, 99.4% GC purity).

### Example 6: Convert Feedstock to DMT Through Glycolysis Using a PET/EG Ratio of 1/1 Followed by Short Path Distillation

A 3-necked 1-liter round-bottom flask was equipped with a mechanical stirrer, a reflux condenser, and a thermocouple. Charge 25% PCR sample (120.13 g), EG (121.7 g) and K₂CO₃ (1.21 g). The resulting mixture was refluxed for 2 hours. After the reaction mixture was cooled to 50° C., 50% aqueous NaOH solution (749 mg) and MeOH (480.0 g) were added. The resulting mixture was kept at 50° C. and stirred for 30 minutes. The flask was allowed to cool to room temperature and product was recovered by filtration and wash. After drying in the air for overnight, DMT product was obtained 102.31 gas grey powder (97.73% GC purity).

A 3-necked 1-liter round bottom flask was equipped with a magnetic stir bar, a heating mantle, a distillation head and an air condenser, and a thermocouple. Both distillation head and air condenser were insulated with a heat tape and a thermocouple. Charge 99.5 g crude DMT. DMT product was collected as white needle (92.49 g, 99.7% GC purity).

### Example 7: Convert Feedstock to DMT Through Glycolysis Using a PET/EG Ratio of 1/1 Followed by Short Path Distillation

A 3-necked 1-liter round-bottom flask was equipped with a mechanical stirrer, a reflux condenser, and a thermocouple. Charge FDST-251 sample (120.01 g), EG (121.35 g) and K₂CO₃ (1.21 g). The resulting mixture was refluxed for 2 hours. After the reaction mixture was cooled to 50° C., 50% aqueous NaOH solution (749 mg) and MeOH (483.17 g) were added.

The resulting mixture was kept at 50° C. and stirred for 30 minutes. The flask was allowed to cool to room temperature and product was recovered by filtration and wash. After drying in the air for overnight, DMT product was obtained 103.01 gas grey powder (92.57% GC purity).

A 3-necked 1-liter round bottom flask was equipped with a magnetic stir bar, a heating mantle, a distillation head and an air condenser, and a thermocouple. Both distillation head and air condenser were insulated with a heat tape and a thermocouple. Charge 99.8 g crude DMT. DMT product was collected as white needle (85.9 g, 99.1% GC purity).

TABLE 2
GC analysis of DMT before and after distillation
(comparing Examples 4-7)

| | Description | wt (g) | EG | DMT | DMI | MHT | MHET | BHET | Unknowns | Total |
|---|---|---|---|---|---|---|---|---|---|---|
| Example 4 | Crude DMT | 32.27 | 0.08 | 85.81 | 0.03 | 0.04 | 0.23 | 0.24 | 0.19 | 88.09 |
| Example 5 | Crude DMT | 90.07 | 0.56 | 95.73 | 0.00 | 0.01 | 0.35 | 0.04 | 0.18 | 97.48 |
| | distilled | 112.15 | 0.27 | 100.29 | 0.00 | 0.00 | 0.03 | 0.00 | 0.18 | 100.80 |
| | DMT | | | | | | | | | |
| Example 6 | Crude DMT | 102.31 | 0.42 | 97.73 | 0.00 | 0.03 | 0.41 | 0.01 | 0.04 | 99.78 |
| | distilled | 92.49 | 0.00 | 101.26 | 0.00 | 0.13 | 0.06 | 0.00 | 0.03 | 101.48 |
| | DMT | | | | | | | | | |
| Example 7 | Crude DMT | 103.01 | 0.32 | 92.57 | 0.00 | 0.00 | 0.27 | 0.02 | 0.38 | 95.75 |
| | Distilled | 85.9 | 0.16 | 99.26 | 0.00 | 0.01 | 0.12 | 0.00 | 0.57 | 100.12 |
| | DMT | | | | | | | | | |

As shown in Table 2, flash distillation significantly improved the quality of DMT as indicated by lower levels of impurities and better total accountable mass. In addition, these processes demonstrated an efficient separation of isophthalate from terephthalate.

### Example 8: Metal Analysis of Distilled DMT and Analysis of Raw Materials

Metal analysis was carried out on distilled DMT product from Examples 5, 6, and 7, as well as on the feedstock raw materials. A description of the metal analysis and of the feedstocks appears above. The results are provided in Tables 3 and 4 below. IPA and TPA we % was determined using LC, as described above.

Low level residual metal was observed in the distilled DMT product when we started with solid-state blend (SSB), 25% PCR, and FDST-251 textile and carpet. As shown in Tables 3 and 4, the processes described herein demonstrated an efficient removal of a variety of elements, including metals (Sb, Ca, Fe, Mn, Na, Ti), halogen (Br, Cl) and other elements (P and S).

TABLE 3
Metal Analysis of distilled DMT

| | Raw | | | | | | | | | | |
|---|---|---|---|---|---|---|---|---|---|---|---|
| Sample | material | Sb | Br | Ca | Cl | Fe | Mn | P | Na | S | Ti |
| Example 5 | SSB | <2 | <5 | <5 | 39.9 | 2.2 | <5 | <2 | <30 | <5 | <5 |
| Example 6 | 25% | <2 | <5 | 7.1 | <10 | 8.9 | <5 | <2 | 40 | <5 | <5 |
| | PCR | | | | | | | | | | |
| Example 7 | FDST- | <2 | <5 | 8.7 | <10 | <2 | <5 | <2 | <30 | <5 | <5 |
| | 251 | | | | | | | | | | |

TABLE 4
Analysis of Raw Material Feedstocks

| Raw | IPA | TPA | | | | | | | | | | |
|---|---|---|---|---|---|---|---|---|---|---|---|---|
| material | wt % | wt % | Sb | Br | Ca | Cl | Fe | Mn | P | Na | S | Ti |
| SSB | 1.59 | 84.2 | 146 | 8.7 | 1947 | 50 | 124 | 10 | 59 | 461 | 78 | 149 |
| 25% | 1.56 | 82.1 | 211 | 6.4 | 182 | 514 | 104 | <5 | 31 | 100 | 14 | 817 |
| PCR | | | | | | | | | | | | |
| FDST | 1.51 | 77.1 | 165 | 22 | 7515 | 234 | 333 | 8 | 163 | 905 | 245 | 557 |
| 251 | | | | | | | | | | | | |

The present disclosure can also be described in accordance with the following numbered clauses.
Clause 1. A process for recovering one or more dialkyl terephthalates from a feedstock composition, comprising: exposing a feedstock composition comprising one or more polyesters and one or more foreign materials to one or more glycols and a depolymerization catalyst in a first reaction vessel under depolymerization conditions to provide a first mixture, the first mixture comprising one or more depolymerization products; exposing at least a portion of the first mixture to an alcohol composition and an alcoholysis catalyst under alcoholysis conditions to provide a second mixture, the second mixture comprising one or more dialkyl terephthalates, wherein the alcoholysis conditions comprise a temperature of from 23° C. to 70° C. for 0.5 hours to 10 hours; and isolating at least a portion of the one or more dialkyl terephthalates from the second mixture.
Clause 2. The process of clause 1, wherein the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.
Clause 3. The process of clauses 1-2, further comprising removing a first portion of the one or more foreign materials from the first reaction vessel.
Clause 4. The process of clauses 2-3, wherein the first portion of the one or more foreign materials are removed from the first reaction vessel via a pump.
Clause 5. The process of clauses 2-4, wherein the first portion of the one or more foreign materials comprises polyolefins, polyethylene, polypropylene, polystyrene, or a combination thereof.
Clause 6. The process of clauses 1-5, wherein the one or more foreign materials are present in the feedstock composition in an amount of from 0.01 wt. % to 50 wt. %, relative to the weight of the one or more polyesters.
Clause 7. The process of clauses 1-6, wherein the isolating at least a portion of one or more dialkyl terephthalates from the second mixture comprises exposing at least a portion of the second mixture to distillation conditions to separate at least a portion of one or more dialkyl terephthalates from a distillation pot residue.
Clause 8. The process of clause 7, wherein the distillation pot residue comprises a second portion of the one or more foreign materials.
Clause 9. The process of clause 8, wherein the second portion of the one or more foreign materials comprises at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.
Clause 10. The process of clauses 1-9, wherein the depolymerization conditions comprise a temperature in a range of about 120° C. to about 260° C., a pressure in a range of about 0.013 atm (0.2 psig) to about 2 atm (30 psig), and a time period in a range of about 0.5 hours to about 10 hours.
Clause 11. The process of clauses 1-10, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether) glycol (PTMG), or a combination thereof.
Clause 12. The process of clauses 1-11, wherein a weight ratio of the one or more glycols to the feedstock composition is in a range of about 1:9 to about 9:1.
Clause 13. The process of clauses 1-12, wherein the depolymerization catalyst comprises a member selected from the group consisting of Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.
Clause 14. The process of clauses 1-13, wherein the one or more polyesters comprises polyethylene terephthalate (PET), 1,4-cyclohexanedimethanol (CHDM)-modified PET, isophthalic acid (IPA)-modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.
Clause 15. The process of clauses 1-14, wherein the one or more polyesters comprises 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in the one or more polyesters is about 100 mole %, and wherein the sum of diacid equivalents in the one or more polyesters is about 100 mole %.
Clause 16. The process of clauses 1-15, wherein at least one of the one or more polyesters has an inherent viscosity of from about 0.1 dL/g to about 1.2 dL/g, as determined according to ASTM D2857-70.
Clause 17. The process of clauses 1-16, wherein at least one of the one or more polyesters are recycled polyesters.
Clause 18. The process of clauses 1-17, wherein the alcohol composition comprises methanol.
Clause 19. The process of clauses 1-18, wherein, the alcoholysis catalyst is present in an amount of from 0.1 wt. % to 20 wt. %, relative to the weight of the one or more polyesters in the feedstock composition.
Clause 20. The process of clause 1-19, wherein the alcoholysis catalyst comprises K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.
Clause 21. The process of clauses 1-20, wherein the at least a portion of the one or more dialkyl terephthalates comprises dimethyl terephthalate (DMT), and wherein the DMT is at least 90% pure.
Clause 22. The process of clauses 1-21, wherein the at least a portion of the one or more dialkyl terephthalates further comprises: dimethyl isophthalate (DMI) in an amount of 1000 ppm or less, or 500 ppm or less; bisphenol A (BPA) in an amount of 1000 ppm or less, or 500 ppm or less; or both.
Clause 23. The process of clauses 1-22, wherein the isolating at least a portion of one or more dialkyl terephthalates from the second mixture comprises exposing the second mixture to a solvent dissolution process.
Clause 24. The process of clauses 1-23, wherein the process is conducted as a batch process, a semi-continuous process, or a continuous process.
Clause 25. The process of clauses 1-24, wherein the one or more depolymerization products comprise monomers, oligomers, or a combination thereof.
Clause 26. The process of clause 25, wherein the one or more oligomers exhibit a degree of polymerization of from 2 to 10.

1. A process for recovering one or more dialkyl terephthalates from a feedstock composition, comprising: exposing a feedstock composition comprising one or more polyesters and one or more foreign materials to one or more glycols and a depolymerization catalyst in a first reaction vessel under depolymerization conditions to provide a first mixture, the first mixture comprising one or more depolymerization products; exposing at least a portion of the first mixture to an alcohol composition and an alcoholysis catalyst under alcoholysis conditions to provide a second mixture, the second mixture comprising one or more dialkyl terephthalates, wherein the alcoholysis conditions comprise a temperature of from 23° C. to 70° C. for 0.5 hours to 10 hours; and isolating at least a portion of the one or more dialkyl terephthalates from the second mixture.

2. The process of claim 1, wherein the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.

3. The process of claim 1, further comprising removing a first portion of the one or more foreign materials from the first reaction vessel.

4. The process of claim 2, wherein the first portion of the one or more foreign materials are removed from the first reaction vessel via a pump.

5. The process of claim 2, wherein the first portion of the one or more foreign materials comprises polyolefins, polyethylene, polypropylene, polystyrene, or a combination thereof.

6. The process of claim 1, wherein the one or more foreign materials are present in the feedstock composition in an amount of from 0.01 wt. % to 50 wt. %, relative to the weight of the one or more polyesters.

7. The process of claim 1, wherein the isolating at least a portion of one or more dialkyl terephthalates from the second mixture comprises exposing at least a portion of the second mixture to distillation conditions to separate at least a portion of one or more dialkyl terephthalates from a distillation pot residue.

8. The process of claim 7, wherein the distillation pot residue comprises a second portion of the one or more foreign materials.

9. The process of claim 8, wherein the second portion of the one or more foreign materials comprises at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.

10. The process of claim 1, wherein the depolymerization conditions comprise a temperature in a range of about 120° C. to about 260° C., a pressure in a range of about 0.013 atm (0.2 psig) to about 2 atm (30 psig), and a time period in a range of about 0.5 hours to about 10 hours.

11. The process of claim 1, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether) glycol (PTMG), or a combination thereof.

12. The process of claim 1, wherein a weight ratio of the one or more glycols to the feedstock composition is in a range of about 1:9 to about 9:1.

13. The process of claim 1, wherein the depolymerization catalyst comprises a member selected from the group consisting of Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.

14. The process of claim 1, wherein the one or more polyesters comprises polyethylene terephthalate (PET), 1,4-cyclohexanedimethanol (CHDM)-modified PET, isophthalic acid (IPA)-modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.

15. The process of claim 1, wherein the one or more polyesters comprises 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in the one or more polyesters is about 100 mole %, and wherein the sum of diacid equivalents in the one or more polyesters is about 100 mole %.

16. The process of claim 1, wherein the alcohol composition comprises methanol.

17. The process of claim 1, wherein, the alcoholysis catalyst is present in an amount of from 0.1 wt. % to 20 wt. %, relative to the weight of the one or more polyesters in the feedstock composition, and wherein the alcoholysis catalyst comprises K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.

18. The process of claim 1, wherein the at least a portion of the one or more dialkyl terephthalates comprises dimethyl terephthalate (DMT), and wherein the DMT is at least 90% pure.

19. The process of claim 1, wherein the isolating at least a portion of one or more dialkyl terephthalates from the second mixture comprises exposing the second mixture to a solvent dissolution process.

20. The process of claim 1, wherein the one or more depolymerization products comprise monomers, oligomers, or a combination thereof, and wherein the one or more oligomers exhibit a degree of polymerization of from 2 to 10.
